(19) United States
(12) Patent Application Publication
(10) Pub. No.: US 2007/0258939 A1
(43) Pub. Date: Nov. 8, 2007

(54) Drug Delivery from Embolic Agents

(75) Inventors: Andrew Lennard Lewis, Surrey (GB); Peter William Stratford, Surrey (GB); Simon William Leppard, Surrey (GB)

Correspondence Address: SUGHRUE MION, PLLC, 2100 PENNSYLVANIA AVENUE, N.W., SUITE 800, WASHINGTON, DC, 20037, US

(73) Assignee: Biocamparibles UK Limited, Surrey (GB)

(21) Appl. No.: 11/573,169

(22) PCT Filed: Aug. 3, 2004
(86) PCT No.: PCT/GB04/03347

§ 371 Date: Jun. 13, 2007

(51) Int. Cl. A61K 31/74

(52) U.S. Cl. 424/78.08

(57) ABSTRACT

A pharmaceutical composition for embolisation of blood vessels, especially for benign tumours, comprises a polymeric embolic agent and, associated with the polymer in a releasable form, a local anaesthetic agent. The polymer is preferably in particulate form, such as in the form of microspheres. A suitable polymer is a crosslinked polyvinyl alcohol polymer formed by the copolymerisation of PVA macromer with other ethylenically unsaturated monomers. The composition provides a synergistic treatment for the symptoms of tumours such as uterine fibroids, leading to size regression as well as pain relief.

The present invention relates to compositions which embolise blood vessels and deliver drugs at the site of embolisation. The drugs are local anaesthetics, to reduce pain.

Embolisation therapy involves the introduction of an agent into the vasculature in order to bring about the deliberate blockage of a particular vessel. This type of therapy is particularly useful for blocking abnormal connections between arteries and veins (such as arteriovenous malformations, or AVMs), and also for occluding vessels that feed certain hyper-vascularised tumours, in order to starve the abnormal tissue and bring about its necrosis and shrinkage. One application of embolotherapy that is receiving increasing attention is the treatment of uterine fibroids. Uterine fibroids or leiomyomata are the most common tumour found in women. Fibroids are benign clonal tumours arising from the smooth-muscle cells of the uterus. Approximately 25% of premenopausal women suffer from fibroids, while the overall prevalence of these tumours could be as high as 77%. The incidence of fibroids in African-American women is three times that of Caucasian women. Fibroids may occur at any age, but are most common in women over the age of 40 years. After menopause, fibroids usually regress in size due to the lack of hormonal stimulation, which may result in infarction.

The rationale for utilizing embolisation to treat uterine fibroids can be traced to several known indications for embolotherapy. First, embolisation has been used with success as a palliative treatment in end-stage cancer patients for symptomatic relief. Examples of this include patients with bony metastases arising from renal cell carcinoma and patients with inoperable liver tumours (hepatoma and colon metastases). The reason why this procedure works in this scenario is because depriving a tumour of its blood supply ultimately decreases the size of the tumour, resulting in relief of mass-related symptoms. Second, embolisation has been shown to reduce the vascularity of tumours prior to surgical excision thereby reducing intraoperative blood loss; this indication has been utilized for renal cell carcinomas and spinal tumours prior to resection. Third, embolisation has been used with success to control tumour-related bleeding in sites throughout the body. Examples of this success include bleeding secondary to renal cell caricinoma, bladder tumours, angiomyolipoma, and hepatic adenomas. Finally, embolisation has been used with success to control abnormal uterine bleeding due to gynecologic malignancies (endometrial, cervical, and ovarian), postpartum bleeding, postsurgical bleeding, bleeding from an ectopic pregnancy and bleeding due to congenital AVMs. A recent article by Vedantham, et al Appl Radiol, 31(10):9-17, 2002, reviews the indications for uterine artery embolization in the obstetrical and gynecologic patient population.

In the major studies of uterine fibroid embolisation to date, the most frequently used embolic material is particulate foamed polyvinyl alcohol, which has been classified according to its particle size. The foam is delivered in suspension form in an aqueous vehicle, using a micocatheter, delivered to one or both of the uterine arteries.

One drawback to the UFE procedure is the associated pain that may be experienced by the patient. For this reason, conscious sedation and analgesia are critical to the successful outcome of a UFE procedure. Not only does this help to reduce anxiety, but more specifically addresses the severe pelvic pain, cramps, and nausea that is termed postembolisation syndrome. Immediately following the UFE procedure, the patient can use an analgesia pump to self-administer narcotic pain relief. Supplementation with systemic analgesics helps to reduce the amount of narcotic used by combating pain and cramping. From four of the trials listed by Vedantham et al, despite high procedural success, pain is encountered as a major result.

Periprocedural pain control therefore, is of utmost importance since it can represent the major morbidity of the procedure. Pain generally starts early after the embolisation and reaches the highest severity 24 to 48 hours after the embolisation. Most pain protocols use a combination of opioids, such as an oxycodone derivative, and a nonsteroidal anti-inflammatory (NSAID), such as ibuprofen or ketorolac. Successful pain control potentially allows this procedure to be performed on an outpatient basis. Early studies attempting to perform UFE as an outpatient procedure reported that 15% of patients returned to the hospital for pain control. One should not use intra-arterial lidocaine in an attempt to reduce pain since it causes a large amount of spasm (Keyoung J A, Levy E B, Roth A R, et al. Intraarterial lidocaine for pain control after uterine artery embolization for leiomyomata. J Vasc Intervent Radiol. 2001; 12:1065-1069). Postembolization syndrome with severe pain, fever, and an elevation in the white blood count occurs in as many as 34% of patients. (Goodwin S C, McLucas B, Lee M, et al. Uterine artery embolization for the treatment of uterine leiomyomata midterm results. Vasc Intervent Radiol. 1999; 10:1159-1165).

Others have described the formulation of microparticles with therapeutic agents. WO 2004039355 describes biodegradable microspheres which optionally contain anaesthetics for intra-articular injection. The particles have sizes less than 100 μm in diameter. The particles are formed by codissolving drug and polymer and forming microspheres from the mixture. The polymer is poly(lactic-glycolic acid). WO2000024378 discloses therapeutic compositions of microspheres for application to wounds. Anaesthetics are mentioned as possible chemotherapeutic agents for incorporating into the compositions. BR9710421 describes skin cosmetic formulation in microsphere form containing lidocaine. WO 2000019976 uses microspheres of a polyester-polyphosphonate or -polyphosphite containing 3.5-4% lidocaine for implantation or injection into the body. The microspheres are small (less than 20 μm) and are produced by a method involving a common solution of drug and polymer. U.S. Pat. No. 6,451,335 describes microparticles of particle size usually less than 100 μm for providing prolonged local anaesthesia. The polymer used is poly(lactic glycolic acid) and the particles are formed by process involving formation of a common solution of polymer and drug. For those cases where microspheres are to be injected into the bloodstream for delivery of drugs thereto, the sizes were typically of the range less than 10 μm, such that they can circulate without causing an embolism.

According to the present invention there is provided a new use of a polymeric embolic agent and, associated with the polymer in a releasable form, a local anaesthetic compound, in the manufacture of a composition for pain relief in the embolisation of blood vessels.

The invention allows local delivery of anaesthetics for pain relief in embolisation via a polymer-based embolic agent. Preferably the polymer is a water-insoluble material. Although it may be biodegradable, so that drug may be released substantially by erosion of polymer matrix to release drug from the surface, preferably the polymer is substantially biostable. It is preferred for the polymer to be water-swellable.

Water-swellable polymer useful in the invention preferably has a equilibrium water content, when swollen in water at 37° C., measured by gravimetric analysis, in the range of 40 to 99 wt %, preferably 75 to 95%.

The polymer may be in the form of a coating on an embolic device such as a metal coil. Preferably, however, the embolic agent is in the form of particles of bulk polymer, or alternatively foamed polymer, having open or closed cells therein. Alternatively, the polymeric agent may be formed in situ, by delivery of a liquid agent and curing at the site of embolisation to form an insoluble polymer matrix.

In the preferred embodiment of the invention, the composition which is administered to a patient in need of embolisation therapy, is in the form of a suspension of particles of water-swollen water-insoluble polymer. Preferably the particles are graded into calibrated size ranges for accurate embolisation of vessels. The particles preferably have sizes in the range 40 to 1500 μm, more preferably in the range 100 to 1200 μm. The calibrated ranges may comprise particles having diameters with a bandwidth of about 100 to 300 μm. The size ranges may be for instance 100 to 300, 300 to 500, 500 to 700 μm, 700 to 900 μm and 900 to 1200 μm. Preferably the particles are substantially spherical in shape. Such particles are referred to herein as microspheres.

Generally the polymer is covalently crosslinked, although it may be appropriate for the polymer to be ionically crosslinked, at least in part. The polymer may be formed by polymerising ethylenically unsaturated monomers in the presence of di- or higher-functional crosslinking monomers, the ethylenically unsaturated monomers preferably including an ionic (including zwitterionic) monomer. Copolymers of hydroxyethyl methacrylate, acrylic acid and cross-linking monomer, such as ethylene glycol dimethacrylate or methylene bisacrylamide, as used for etafilcon A based contact lenses may be used.

Polymer microspheres used as ion-exchange media which have been utilised as embolic agents may also be used. These are, for instance, based on cross-linked acrylic polymers, cross-linked polyvinylalcohol, or most commonly, cross-linked styrene divinyl benzene copolymers, all with appropriate functionalities to confer ion-exchange capabilities.

Another type of polymer which may be used to form the water-swellable water-insoluble matrix is polyvinyl alcohol crosslinked using aldehyde type crosslinking agents such as glutaraldehyde. For such products, the polyvinyl alcohol (PVA) may be rendered ionic. For instance the PVA may be rendered ionic by providing pendant ionic groups by reacting a functional ionic group containing compound with the hydroxyl groups. Examples of suitable functional groups for reaction with the hydroxyl groups are acylating agents, such as carboxylic acids or derivatives thereof, or other acidic groups which may form esters.

The invention is of particular value where the polymer matrix is formed of a polyvinyl alcohol macromer, having more than one ethylenically unsaturated pendant group per molecule, by radical polymerisation of the ethylenic groups. Preferably the PVA macromer is copolymerised with ethylenically unsaturated monomers for instance including a nonionic or ionic monomer.

The PVA macromer may be formed, for instance, by providing PVA polymer, of a suitable molecular weight such as in the range 1000 to 500,000 D, preferably 10,000 to 100,000 D, with pendant vinylic or acrylic groups. Pendant acrylic groups may be provided, for instance, by reacting acrylic or methacrylic acid with PVA to form ester linkages through some of the hydroxyl groups. Methods for attaching vinylic groups capable of polymerisation onto polyvinyl alcohol are described in, for instance, U.S. Pat. No. 4,978,713 and, preferably, U.S. Pat. Nos. 5,508,317 and 5,583,163. Thus the preferred macromer comprises a backbone of polyvinyl alcohol to which is linked, via a cyclic acetal linkage, an (alk)acrylaminoalkyl moiety. Example 1 describes the synthesis of such a macromer. Preferably the PVA macromers have about 2 to 20 pendant ethylenic groups per molecule, for instance 5 to 10.

Where PVA macromers are copolymerised with ethylenically unsaturated monomers including an ionic monomer, the ionic monomer preferably has the general formula I
Y¹BQ
in which Y¹ is selected from 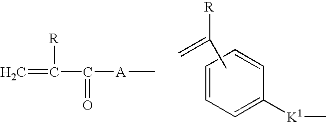
CH₂═C(R)—CH₂—O—, CH₂═C(R)—CH₂OC(O)—, CH₂═C(R)OC(O)—, CH₂═C(R)—O—, CH₂═C(R)CH₂OC(O)N(R¹)—, R²OOCCR═CRC(O)—O—, RCH═CHC(O)O—, RCH═C(COOR²)CH₂—C(O)—O—, 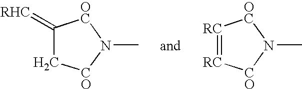
wherein:

R is hydrogen or a C₁-C₄ alkyl group;

R¹ is hydrogen or a C₁-C₄ alkyl group;

R² is hydrogen or a C₁₋₄ alkyl group or BQ where B and Q are as defined below;

A is —O— or —NR¹—;

K¹ is a group —(CH₂)ᵣOC(O)—, —(CH₂)ᵣC(O)O—, —(CH₂)ᵣOC(O)O—, —(CH₂)ᵣNR³—, —(CH₂)ᵣNR³C(O)—, —(CH₂)ᵣC(O)NR³—, —(CH₂)ᵣNR³C(O)O—, —(CH₂)ᵣOC(O)NR³—, —(CH₂)ᵣNR³C(O)NR³— (in which the groups R³ are the same or different), —(CH₂)ᵣO—, —(CH₂)ᵣSO₃—, or, optionally in combination with B¹, a valence bond and r is from 1 to 12 and R³ is hydrogen or a C₁-C₄ alkyl group;

B is a straight or branched alkanediyl, oxaalkylene, alkanediyloxaalkanediyl, or alkanediyloligo(oxaalkanediyl) chain optionally containing one or more fluorine atoms up to and including perfluorinated chains or, if Q or Y¹ contains a terminal carbon atom bonded to B a valence bond; and

Q is an ionic group.

An anionic group Q may be, for instance, a carboxylate, carbonate, sulphonate, sulphate, nitrate, phosphonate or phosphate group. The monomer may be polymerised as the free acid or in salt form. Preferably the pKₐ of the conjugate acid is less than 5.

A suitable cationic group Q is preferably a group N⁺R⁴₃, P⁺R⁵₃ or S⁺R⁵₂

in which the groups R⁴ are the same or different and are each hydrogen, C₁₋₄-alkyl or aryl (preferably phenyl) or two of the groups R⁴ together with the heteroatom to which they are attached from a saturated or unsaturated heterocyclic ring containing from 5 to 7 atoms the groups R⁵ are each OR⁴ or R⁴. Preferably the cationic group is permanently cationic, that is each R⁴ is other than hydrogen. Preferably a cationic group Q is N⁺R⁴₃ in which each R⁴ is C₁₋₄-alkyl, preferably methyl.

A zwitterionic group Q may have an overall charge, for instance by having a divalent centre of anionic charge and monovalent centre of cationic charge or vice-versa or by having two centres of cationic charge and one centre of anionic charge or vice-versa. Preferably, however, the zwitterion has no overall charge and most preferably has a centre of monovalent cationic charge and a centre of monovalent anionic charge.

Examples of zwitterionic groups which may be used as Q in the present invention are disclosed in WO-A-0029481.

Where the ethylenically unsaturated monomer includes zwitterionic monomer, for instance, this may increase the hydrophilicity, lubricity, biocompatibility and/or haemocompatibility of the particles. Suitable zwitterionic monomers are described in our earlier publications WO-A-9207885, WO-A-9416748, WO-A-9416749 and WO-A-9520407. Preferably a zwitterionic monomer is 2-methacryloyloxy-2′-trimethylammonium ethyl phosphate inner salt (MPC).

In the monomer of general formula I preferably Y¹ is a group CH₂═CRCOA— in which R is H or methyl, preferably methyl, and in which A is preferably NH. B is preferably an alkanediyl group of 1 to 12, preferably 2 to 6 carbon atoms.

There may be included in the ethylenically unsaturated monomer diluent monomer, for instance non-ionic monomer. Such monomer may be useful to control the pKₐ of the acid groups, to control the hydrophilicity or hydrophobicity of the product, to provide hydrophobic regions in the polymer, or merely to act as inert diluent. Examples of non-ionic diluent monomer are, for instance, alkyl(alk) acrylates and (alk) acrylamides, especially such compounds having alkyl groups with 1 to 12 carbon atoms, hydroxy, and di-hydroxy-substituted alkyl(alk) acrylates and -(alk) acrylamides, vinyl lactams, styrene and other aromatic monomers.

In the polymer matrix, where there is ionic group present the level of ion is preferably in the range 0.1 to 10 meq g⁻¹, preferably at least 1.0 meq

Where PVA macromer is copolymerised with other ethylenically unsaturated monomers, the weight ratio of PVA macromer to other monomer is preferably in the range of 50:1 to 1:5, more preferably in the range 20:1 to 1:2. In the ethylenically unsaturated monomer the ionic monomer is preferably present in an amount in the range 10 to 100 mole %, preferably at least 25 mole %.

The polymer may be formed into particles in several ways. For instance, the crosslinked polymer may be made as a bulk material, for instance in the form of a sheet or a block, and subsequently be comminuted to the desired size. Alternatively, the crosslinked polymer may be formed as such in particulate form, for instance by polymerising in droplets of monomer in a dispersed phase in a continuous immiscible carrier. Examples of suitable water-in-oil polymerisations to produce particles having the desired size, when swollen, are known. For instance U.S. Pat. No. 4,224,427 describes processes for forming uniform spherical beads (microspheres) of up to 5 mm in diameter, by dispersing water-soluble monomers into a continuous solvent phase, in a presence of suspending agents. Stabilisers and surfactants may be present to provide control over the size of the dispersed phase particles. After polymerisation, the crosslinked microspheres are recovered by known means, and washed and optionally sterilised. Preferably the particles eg microspheres, are swollen in an aqueous liquid, and classified according to their size.

Other examples of suitable polymeric embolic agents are foamed polyvinylalcohol, foamed gelatin, gelatin, alginates, starches, celluloses or other polysaccharides or collagen cross-linked with aldehydes or other di- or higher-functional reagents, tris-acryl copolymers cross-linked by collagen or gelatin, silk, polymers formed in situ from cyano-based surgical adhesives, ethylene-vinyl acetate polymers dissolved in DMSO and precipitated in situ in the blood vessel, etc.

Examples of specific agents useful in the present invention are benzocaine, bupivacaine, chloroprocaine, etidocaine, lidocaine, lignocaine, mepivacaine, novacaine, prilocalne, procaine, tetracaine, butacaine, carticaine, fomocaine, isobucaine, ketamine, leucinocaine, meprylcaine, myrtecaine, octacaine, oxybuprocaine, parethoxycaine, phenacaine, piperocaine, pramoxine, propanocaine propoxycaine, proxymethacaine, pyrrocaine, ropivicaine, tolycaine and xyolcaine. Particularly preferred are benzocaine, bupivacaine, chloroprocaine, etidocaine, lidocaine, lignocaine, mepivacaine, novacaine, prilocalne, procaine and tetracaine, more preferably lidocaine or procaine.

The pharmaceutical agent is associated with the polymer preferably so as to allow controlled release of the agent over a period. Where the agent is for pain relief this period may be up to a few days, preferably up to 72 hours when most postoperative pain is experienced. The agent may be electrostatically, or covalently bonded to the polymer or held by Van der Waal's interactions.

The pharmaceutical active may be incorporated into the polymer matrix by a variety of techniques. In one method, the active may be mixed with a precursor of the polymer, for instance a monomer or macromer mixture or a cross-linkable polymer and cross-linker mixture, prior to polymerising or crosslinking. Alternatively, the active may be loaded into the polymer after it has been crosslinked. For instance, particulate dried polymer may be swollen in a solution of active, preferably in water, optionally with subsequent removal of non-absorbed agent and/or evaporation of solvent. A solution of the active, in an organic solvent such as an alcohol, or, more preferably, in water, may be sprayed onto a moving bed of particles, whereby drug is absorbed into the body of the particles with simultaneous solvent removal. Most conveniently, we have found that it is possible merely to contact swollen polymer particles suspended in a continuous liquid vehicle, such as water, with a solution of drug, over a period, whereby drug becomes absorbed into the body of the particles. The swelling vehicle may subsequently be removed or, conveniently, may be retained with the particles as part of the product for subsequent use as an embolic agent or the swollen particles may be used in swollen form in the form of a slurry, i.e. without any or much liquid outside the swollen particles.

Alternatively, the suspension of particles can be removed from any remaining drug loading solution and the particles dried by any of the classical techniques employed to dry pharmaceutical-based products. This could include, but is not limited to, air drying at room or elevated temperatures or under reduced pressure or vacuum; classical freeze-drying; atmospheric pressure-freeze drying; solution enhanced dispersion of supercritical fluids (SEDS). Alternatively drug-loaded microspheres may be dehydrated using an organic solvent to replace water in a series of steps, followed by evaporation of the more volatile organic solvent. A solvent should be selected which is a non-solvent for the drug.

In brief, a typical classical freeze drying process might proceed as follows: the sample is aliquoted into partially stoppered glass vials, which are placed on a cooled, temperature controlled shelf within the freeze dryer. The shelf temperature is reduced and the sample is frozen to a uniform, defined temperature. After complete freezing, the pressure in the dryer is lowered to a defined pressure to initiate primary drying. During the primary drying, water vapour is progressively removed from the frozen mass by sublimation whilst the shelf temperature is controlled at a constant, low temperature. Secondary drying is initiated by increasing the shelf temperature and reducing the chamber pressure further so that water absorbed to the semi-dried mass can be removed until the residual water content decreases to the desired level. The vials can be sealed, in situ, under a protective atmosphere if required.

Atmospheric pressure freeze drying is accomplished by rapidly circulating very dry air over a frozen product. In comparison with the classical freeze-drying process, freeze-drying without a vacuum has a number of advantages. The circulating dry gas provides improved heat and mass transfer from the frozen sample, in the same way as washing dries quicker on a windy day. Most work in this area is concerned with food production, and it has been observed that there is an increased retention of volatile aromatic compounds, the potential benefits of this to the drying of biologicals is yet to be determined. Of particular interest is the fact that by using atmospheric spray drying processes instead of a cake, a fine, free-flowing powder is obtained. Particles can be obtained which have submicron diameters, this is tenfold smaller than can be generally obtained by milling. The particulate nature, with its high surface area results in an easily rehydratable product.

According to a further aspect of the invention there is provided a new pharmaceutical composition comprising particles of water-insoluble water-swellable polymer having average particle size when swollen in distilled water to equilibrium at 37° C. in the range 100 to 1500 μm and an equilibrium water content in the range 45 to 99% by weight and a local anaesthetic agent.

The particle sizes are generally determined in the absence of the local anaesthetic. Preferably the particles comprise a fraction of particles separated from a population, having sizes with a bandwidth in the range 100 to 300 μm, more preferably provided in the form of at least two compositions, each having particles of different fractions, which preferably do not substantially overlap. Such fractions are generally formed by fractionation of a population with a wide particle size distribution on the basis of size, for instance using sieves with appropriate sized apertures.

The particles of this aspect of the invention preferably have the features described above in relation to the first aspect of the invention.

The composition may be provided for use in dry form such that rehydration is carried out immediately prior to administration. In some instances the novel compositions may be formulated immediately before use by mixing separately supplied embolic agent and anaesthetic. Preferably, however the embolic agent and local anaesthetic agent are premixed, as described above.

Preferably the anaesthetic is selected from benzocaine, bupivacaine, chloroprocaine, etidocaine, lidocaine, lignocaine, mepivacaine, novacaine, prilocalne, procaine, tetracaine butacaine, carticaine, fomocaine, isobucaine, ketamine, leucinocaine, meprylcaine, myrtecaine, octacaine, oxybuprocaine, parethoxycaine, phenacaine, piperocaine, pramoxine, propanocaine propoxycaine, proxymethacaine, pyrrocaine, ropivicaine, tolycaine and xyolcaine.

Suitably the pharmaceutical composition comprises an injectable liquid which is absorbed into the particles and is present in an excess of the amount absorbable into the particles. In such compositions the anaesthetic is preferably substantially all adsorbed on or absorbed in the particles, although some may be dissolved in the excess liquid. The liquid is suitably physiological saline. The compositions may comprise imaging agent, or imaging agent may be admixed immediately before delivery.

According to a further aspect of the invention there is provided a method of forming the novel compositions in which particles of water-insoluble water-swellable polymer are contacted with a solution of the local anaesthetic agent in a solvent in which the polymer is swellable whereby the local anaesthetic agent is adsorbed onto or absorbed into the particles of polymer.

Preferably in the method the solvent comprises water although organic solvents which are acceptable and/or may be removed during processing may be utilised. The method may involve removal of a portion or all of the solvent after the initial contact step. Preferred embodiments of the method are described above and in the worked examples below.

The embolic composition is administered in the normal manner for embolisation of blood vessels. Thus the composition may be admixed immediately before administration by the interventional radiologist, with imaging agents such as radiopaque agents. Alternatively or additionally, the particles may be preloaded with radiopaque material in addition to the local anaesthetic agent. Thus the polymer and local anaesthetic agent, provided in preformed admixture, may be mixed with a radiopaque imaging agent in a syringe, used as the reservoir for the delivery device. The composition may be administered, for instance, from a microcatheter device, into the selected arteries. The invention is of use for embolising tumours, especially benign tumours. Selection of suitable particle size range, dependent upon the desired site of embolisation may be made in the normal way by the interventional radiologists. The invention is of particular value in uterine fibroid embolisation, which is associated with pain in the immediate post-operative period.

Intra arterial doses of anaesthetic, e.g. lidocaine, are typically in the range 1-7 mg/kg, not exceeding 500 mg total. The doses locally delivered from polymer in the invention might typically range from 0.1 to 100 mg/ml composition administered, with a volume of composition administered typically being in the range 0.1 to 5 ml, preferably 1 to 2 ml.

The example is illustrated in the following examples and figures, in which:

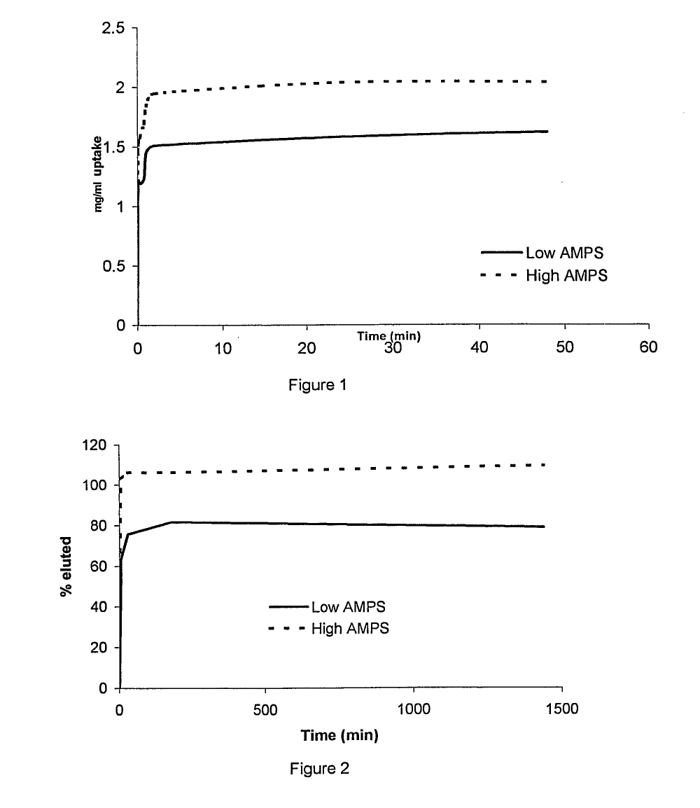

FIG. 1 shows the loading profile for the experiment described in example 2;

FIG. 2 shows the release profile for the experiment of example 2.

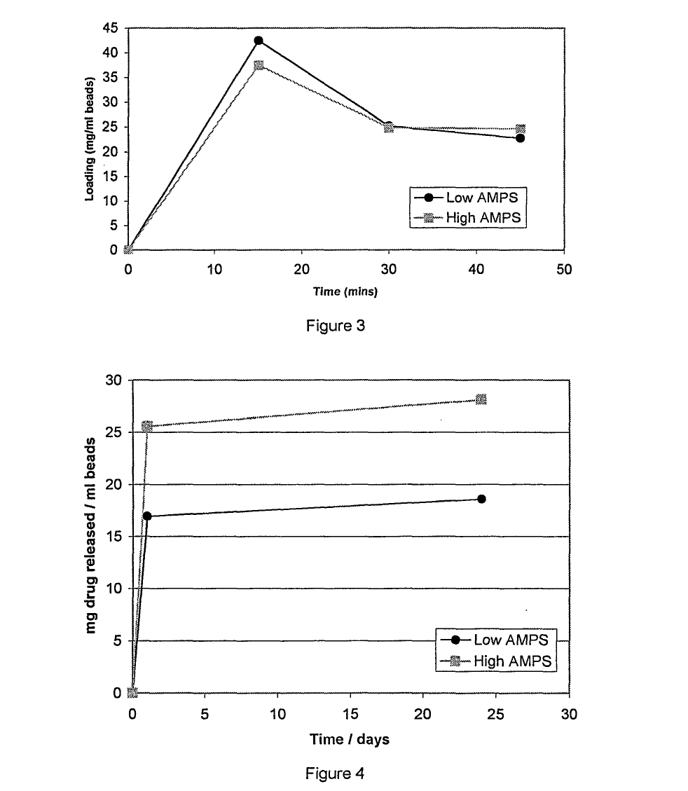

FIGS. 3 and 4 show the loading and release profile results, respectively, for Example 3.

### EXAMPLE 1

### Outline Method for the Preparation of Microspheres

Nelfilcon B Macromer Synthesis:

The first stage of microsphere synthesis involves the preparation of Nelfilcon B—a polymerisable macromer from the widely used water soluble polymer PVA. Mowiol 8-88 poly(vinyl alcohol) (PVA) powder (88% hydrolised, 12% acetate content, average molecular weight about 67,000D) (150 g) (Clariant, Charlotte, N.C. USA) is added to a 21 glass reaction vessel. With gentle stirring, 1000 ml water is added and the stirring increased to 400 rpm. To ensure complete dissolution of the PVA, the temperature is raised to 99±9° C. for 2-3 hours. On cooling to room temperature N-acryloylaminoacetaldehyde (NAAADA) (Ciba Vision, Germany) (2.49 g or 0.104 mmol/g of PVA) is mixed in to the PVA solution followed by the addition of concentrated hydrochloric acid (100 ml) which catalyses the addition of the NAAADA to the PVA by transesterification. The reaction proceeds at room temperature for 6-7 hours then stopped by neutralisation to pH 7.4 using 2.5M sodium hydroxide solution. The resulting sodium chloride plus any unreacted NAAADA is removed by diafiltration (step 2).

Diafiltration of Macromer:

Diafiltration (tangential flow filtration) works by continuously circulating a feed solution to be purified (in this case nelfilcon B solution) across the surface of a membrane allowing the permeation of unwanted material (NaCl, NAAADA) which goes to waste whilst having a pore size small enough to prevent the passage of the retentate which remains in circulation.

Nelfilcon B diafiltration is performed using a stainless steel Pellicon 2 Mini holder stacked with 0.1 m² cellulose membranes having a pore size with a molecular weight cut off of 3000 (Millipore Corporation, Bedford, Mass. USA). Mowiol 8-88 has a weight average molecular weight of 67000 and therefore has limited ability to permeate through the membranes.

The flask containing the macromer is furnished with a magnetic stirrer bar and placed on a stirrer plate. The solution is fed in to the diafiltration assembly via a Masterflex LS peristaltic pump fitted with an Easy Load II pump head and using LS24 class VI tubing. The Nelfilcon is circulated over the membranes at approximately 50 psi to accelerate permeation. When the solution has been concentrated to about 1000 ml the volume is kept constant by the addition of water at the same rate that the filtrate is being collected to waste until 6000 ml extra has been added. Once achieved, the solution is concentrated to 20-23% solids with a viscosity of 1700-3400 cP at 25° C. Nelfilcon is characterised by GFC, NMR and viscosity.

Microsphere Synthesis:

The spheres are synthesised by a method of suspension polymerisation in which an aqueous phase (nelfilcon B) is added to an organic phase (butyl acetate) where the phases are immiscible. By employing rapid mixing the aqueous phase can be dispersed to form droplets, the size and stability of which can be controlled by factors such as stirring rates, viscosity, ratio of aqueous/organic phase and the use of stabilisers and surfactants which influence the interfacial energy between the phases. Two series of microspheres are manufactured, a low AMPS and a higher AMPS series, the formulation of which are shown below.

| | |
|---|---|
| A | High AMPS: |
| Aqueous: | ca 21% w/w Nelfilcon B solution (400 ± 50 g approx) |
| | ca 50% w/w 2-acrylamido-2-methylpropanesulphonate Na salt |
| | (140 ± 10 g) |
| | Purified water (137 ± 30 g) |
| | Potassium persulphate (5.22 ± 0.1 g) |
| | Tetramethyl ethylene diamine TMEDA (6.4 ± 0.1 ml) |
| Organic: | n-Butyl acetate (2.7 ± 0.3 L) |
| | 10% w/w cellulose acetate butyrate in ethyl acetate |
| | (46 ± 0.5 g) |
| | Purified water (19.0 ± 0.5 ml) |
| B | Low AMPS: |
| Aqueous: | ca 21% w/w Nelfilcon B solution (900 ± 100 g approx) |
| | ca 50% w/w 2-acryamido-2-methylpropanesulphonate Na salt |
| | (30.6 ± 6 g) |
| | Purified water (426 ± 80 g) |
| | Potassium persulphate (20.88 ± 0.2 g) |
| | TMEDA (25.6 ± 0.5 ml) |
| Organic: | n-Butyl acetate (2.2 ± 0.3 L) |
| | 10% w/w cellulose acetate butyrate (CAB) in ethyl acetate |
| | (92 ± 1.0 g) |
| | Purified water (16.7 ± 0.5 ml) |

A jacketed 4000 ml reaction vessel is heated using a computer controlled bath (Julabo PN 9-300-650) with feedback sensors continually monitoring the reaction temperature.

The butyl acetate is added to the reactor at 25° C. followed by the CAB solution and water. The system is purged with nitrogen for 15 minutes before the PVA macromer is added. Cross linking of the dispersed PVA solution is initiated by the addition of TMEDA and raising the temperature to 55° C. for three hours under nitrogen. Crosslinking occurs via a redox initiated polymerisation whereby the amino groups of the TMEDA react with the peroxide group of the potassium persulphate to generate radical species. These radicals then initiate polymerisation and crosslinking of the double bonds on the PVA and AMPS transforming the dispersed PVA-AMPS droplets into insoluble polymer microspheres. After cooling to 25° C. the product is transferred to a filter reactor for purification where the butyl acetate is removed by filtration followed by:
Wash with 2×300 ml ethyl acetate to remove butyl acetate and CAB
Equilibrate in ethyl acetate for 30 mins then filtered
Wash with 2×300 ml ethyl acetate under vacuum filtration
Equilibrate in acetone for 30 mins and filter to remove ethyl acetate, CAB and water
Wash with 2×300 ml acetone under vacuum filtration
Equilibrate in acetone overnight
Wash with 2×300 ml acetone under vacuum
Vacuum dry, 2 hrs, 55° C. to remove residual solvents.

Sieving:

The manufactured microsphere product ranges in size from 100 to 1200 microns and must undergo fractionation through a sieving process using a range of mesh sizes to obtain the nominal distributions listed below.
1. 100-300 μm
2. 300-500 μm
3. 500-700 μm
4. 700-900 μm
5. 900-1200 μm

Prior to sieving the spheres are vacuum dried to remove any solvent then equilibrated at 60° C. in water to fully re-hydrate. The spheres are sieved using a 316L stainless steel vortisieve unit (MM Industries, Salem Ohio) with 15″ stainless steel sieving trays with mesh sizes ranging from 32 to 1000 μm. Filtered saline is recirculated through the unit to aid fractionation. Spheres collected in the 32 micron sieve are discarded. The microspheres have an equilibrium water content when swollen in distilled water at 37° C. (measured gravimetrically) of 90% for the low AMPS and 94% for the High AMPS sphere.

### EXAMPLE 2

### Lidocaine

2.1 Loading

For this experiment unsterilised High AMPS and low AMPS microspheres made using the general procedure of Example 1 were used. 0.25 ml of each type of microsphere suspension was transferred to 4, 1 ml syringes. Two of these samples were used for the experiment and the others two as controls. 5 ml of 1.25 mg/ml lidocaine/PBS solution was added to small-glass containers. From this solution a standard curve was produced.

The contents of two syringes were expelled into the drug solution and the contents of the other two syringes into PBS to be used as control and timing was started. The containers were placed on the rotamix and they remained there for the whole experiment.

At predetermined times points (0, 0.08, 0.25, 0.75, 1, 2, 24 and 48 hours) 1 ml of the solution (supernatant) was removed, read and then placed back in the container, so the volume remained constant. Samples were read at 250 nm and concentrations were calculated from the equation of the lidocaine standard curve given above. From the data the mg of drug uptake per 1 ml of beads were calculated and the graph shown in FIG. 1 was plotted.

2.2 Elution

For this experiment the microspheres loaded in Example 2.1 above were used. 0.15 ml of the microspheres were transferred to 1 ml syringes. The content of the syringes were expelled into 10 ml of PBS placed into small glass containers.

When the contents of the syringes were expelled the timing was started. The containers were placed on the rotamix and they remained there for the whole experiment. At predetermined times points (0, 5, 30, 180, 1440 min) 1 ml of the solution (supernatant) was removed, read and then placed back in the container, so the volume remained constant.

Samples were read at 250 nm and concentrations were calculated from the equation of the lidocaine standard curve given above. We used the readings of the PBS as time zero. From the data the percentage of drug eluted per 1 ml of microspheres were calculated and the graph shown in FIG. 2 was plotted.

### EXAMPLE 3

Example 2 was repeated using a 2 ml volume of 30 mg/ml procaine HCl in water in place of lidocaine. The loading and release profiles are shown in FIGS. 3 and 4, respectively.

1. Use of a polymeric embolic agent and, associated with the polymer in a releasable form, a local anaesthetic agent, in the manufacture of a composition for pain relief in the embolisation of blood vessels.

2. Use according to claim 1, in which the polymer is in the form of water-insoluble water-swellable particles, preferably substantially spherical in shape.

3. Use according to claim 2 in which the particles have particle sizes when swollen to equilibrium in water at 37° C. in the range 40 to 1500 μm, preferably 100 to 1200 μm.

4. Use according to any preceding claim in which the polymer is synthetic and biostable.

5. Use according to any preceding claim in which the polymer is cross-linked.

6. Use according to claim 5 in which the polymer is covalently cross-linked.

7. Use according to any preceding claim in which the polymer is formed by the radical polymerisation of poly(vinyl alcohol) macromer having pendant ethylenically unsaturated groups.

8. Use according to claim 7 in which the macromer is copolymerised with ethylenically unsaturated comonomer, preferably an ionic comonomer.

9. Use according to any preceding claim in which the local anaesthetic is selected from benzocaine, bupivacaine, chloroprocaine, etidocaine, lidocaine, lignocaine, mepivacaine, novacaine, prilocalne, procaine, tetracaine, butacaine, carticaine, fomocaine, isobucaine, ketamine, leucinocaine, meprylcaine, myrtecaine, octacaine, oxybuprocaine, parethoxycaine, phenacaine, piperocaine, pramoxine, propanocaine propoxycaine, proxymethacaine, pyrrocaine, ropivicaine, tolycaine and xyolcaine.

10. Use according to any preceding claim in which the embolisation is of tumours.

11. Use according to claim 10 in which the tumours are benign tumours, preferably uterine fibroids.

12. A pharmaceutical composition comprising particles of water-insoluble water-swellable polymer having average particle size when swollen in distilled water to equilibrium at 37° C. in the range 100 to 1500 μm and equilibrium water content in the range 45 to 99% and a local anaesthetic agent.

13. A composition according to claim 12 in which the polymer is as defined in any of claims 4 to 8.

14. A composition according to claim 12 or 13 in which the particles comprise a fraction of particles separated from a population, having sizes with a bandwidth in the range 100 to 300 μm.

15. A product comprising at least two compositions according to claim 14, each having particles of different fractions, which preferably do not substantially overlap.

16. A product according to any of claims 12 to 15 comprising an injectable liquid which is absorbed into the particles and is present in an excess of the amount absorbable into the particles.

17. A product according to claim 16 in which the anaesthetic is adsorbed on or absorbed in the particles.

18. A product according to any of claims 12 to 17 in which the anaesthetic is selected from benzocaine, bupivacaine, chloroprocaine, etidocaine, lidocaine, lignocaine, mepivacaine, novacaine, prilocalne, procaine and tetracaine, butacaine, carticaine, fomocaine, isobucaine, ketamine, leucinocaine, meprylcaine, myrtecaine, octacaine, oxybuprocaine, parethoxycaine, phenacaine, piperocaine, pramoxine, propanocaine propoxycaine, proxymethacaine, pyrrocaine, ropivicaine, tolycaine and xyolcaine.

19. A product according to any of claims 12 to 18 which additionally comprises an imaging agent, preferably a radiopaque material.

20. A method of forming a composition according to claim 12 in which particles of water-insoluble water-swellable polymer are contacted with a solution of the local anaesthetic agent in a solvent in which the polymer is swellable whereby the local anaesthetic agent is adsorbed onto or absorbed into the particles of polymer.

21. A method according to claim 20 in which at least a portion of the said solvent is separated from the particles.

22. A method according to claim 20 or claim 21 in which the solvent comprises water.

23. A method according to any of claims 20 to 22 in which the polymer is defined in any of claims 4 to 8.

24. A method according to any of claims 20 to 23 in which the particles comprise a fraction of particles separated from a population, having sizes with a bandwidth in the range 100 to 300 μm.

25. A method according to any of claims 20 to 24 in which the anaesthetic is selected from benzocaine, bupivacaine, chloroprocaine, etidocaine, lidocaine, lignocaine, mepivacaine, novacaine, prilocalne, procaine, tetracaine, butacaine, carticaine, fomocaine, isobucaine, ketamine, leucinocaine, meprylcaine, myrtecaine, octacaine, oxybuprocaine, parethoxycaine, phenacaine, piperocaine, pramoxine, propanocaine propoxycaine, proxymethacaine, pyrrocaine, ropivicaine, tolycaine and xyolcaine.
